(19) United States
(12) Patent Application Publication
(10) Pub. No.: US 2026/0341511 A1
(43) Pub. Date: Oct. 29, 2026

(54) PREDICTING SINGLE-CELL SPATIAL GENOMICS FROM PATHOLOGY IMAGES FOR PATIENT STRATIFICATION

(71) Applicant: NEC Laboratories America, Inc., Princeton, NJ (US)

(72) Inventors: Jonathan Warrell, Princeton, NJ (US); Renqiang Min, Princeton, NJ (US); Eric Cosatto, Red Bank, NJ (US); Tianci Song, Eagan, MN (US)

(21) Appl. No.: 19/656,212

(22) Filed: Apr. 23, 2026

Related U.S. Application Data
(60) Provisional application No. 63/795,767, filed on Apr. 28, 2025.

(51) Int. Cl. G16H 50/20; G06N 3/042; G06N 3/082; G16H 50/70

(52) U.S. Cl. CPC G16H 50/20; G06N 3/042; G06N 3/082; G16H 50/70

(57) ABSTRACT

Systems and methods for predicting single-cell spatial genomics from pathology images for patient stratification. The systems and methods include categorizing cell types of individual cells in an image based on cell features and assigning vectors to the cell features, connecting cells of a same type within a first distance based on the vectors, and forming a spatial adjacency graph from the connected cells, wherein edges of the spatial adjacency graph are associated with the cell features. The systems and methods further include generating a clinical response prediction to a treatment from the spatial adjacency graph and performing health care actions in response to the clinical response prediction.

### RELATED APPLICATION INFORMATION

This application claims priority to U.S. Provisional Patent App. No. 63/795,767, filed on Apr. 28, 2025, incorporated herein by reference in its entirety.

### BACKGROUND

### Technical Field

The present invention relates to artificial intelligence applications in pathology, and more particularly computer vision techniques to analyze spatial genomics.

### Description of the Related Art

Existing spatial genomics methods can learn biomarkers from a variety of sources, including ground-truth spatial transcriptomics (e.g., Digital Spatial Profiling (DSP) data), imputed bulk transcriptomics, and tile-based methods that infer spatial transcriptomics from hematoxylin and eosin (H&E) images.

Methods based on imputed bulk transcriptomics lack single-cell resolution, which introduces confounding due to variation in cell-type composition and reduces interpretability of learned biomarkers. Tile-based methods similarly operate at an aggregated level, using features extracted from image tiles to impute bulk expression per tile. As a result, these methods cannot resolve single-cell spatial expression patterns and instead aggregate signals across multiple cell types, thereby obscuring cell-type-specific expression.

More generally, such approaches fail to adequately model spatial relationships between tiles and do not account for interactions among cells within a tile. Consequently, these methods do not capture spatial context or cell-cell interactions, which are often predictive of biological function and clinical outcomes.

Other methods demonstrate that informative biomarkers can be learned directly from DSP data and may outperform bulk-derived biomarkers. However, these methods operate on predefined spatial compartments and therefore do not achieve single-cell resolution. This limitation can conflate signals from multiple cell types, obscure rare but clinically relevant cellular subpopulations, and reduce the specificity of identified biomarkers. Additionally, these approaches do not incorporate imputed spatial transcriptomics during training, thereby limiting their ability to leverage data from patients for whom H&E image data is available but spatial genomics data is not. This reduces the robustness and generalizability of the resulting models.

More advanced spatial transcriptomic learning methods utilize adaptive graph neural networks (GNNs) to model spatial relationships between tiles and identify informative tumor and tumor microenvironment (TME) regions. DSP data can be used to train such GNNs to impute cell-type-specific spatial transcriptomics for biomarker discovery and patient stratification. However, these approaches still do not enable imputation at single-cell resolution, resulting in continued aggregation of heterogeneous cellular signals and limiting the ability to capture fine-grained biological variation.

Furthermore, these approaches do not support joint training of imputation and response prediction models, which can lead to misalignment between imputed representations and downstream predictive tasks and propagation of imputation errors. In addition, they do not impose sparsity constraints on learned biomarkers, reducing interpretability, increasing susceptibility to overfitting, and limiting clinical applicability.

### SUMMARY

According to an aspect of the present invention, a method is provided for predicting single-cell spatial genomics from pathology images for patient stratification. The method includes categorizing cell types of individual cells in an image based on cell features and assigning vectors to the cell features, connecting cells of a same type within a first distance based on the vectors, and forming a spatial adjacency graph from the connected cells, wherein edges of the spatial adjacency graph are associated with the cell features. The method also includes generating a clinical response prediction to a treatment from the spatial adjacency graph and performing health care treatment actions in response to the clinical response prediction.

According to another aspect of the present invention, a system is provided for predicting single-cell spatial genomics from pathology images for patient stratification. The system includes a processor and a memory storing computer-readable instructions. The computer-readable instructions when executed by the processor, cause the processor to categorize cell types of individual cells in an image based on cell features and assigning vectors to the cell features, connect cells of a same type within a first distance based on the vectors, and form a spatial adjacency graph from the connected cells, wherein edges of the spatial adjacency graph are associated with the cell features. The computer-readable instructions also cause the processor to generate a clinical response prediction to a treatment from the spatial adjacency graph and perform health care treatment actions in response to the clinical response prediction.

In yet another aspect of the present invention, a computer program product including a non-transitory computer-readable storage medium containing computer program code, the computer program code when executed by one or more processors causes the one or more processors to perform operations. The computer program code including instructions to categorize cell types of individual cells in an image based on cell features and assigning vectors to the cell features, connect cells of a same type within a first distance based on the vectors, and form a spatial adjacency graph from the connected cells, wherein edges of the spatial adjacency graph are associated with the cell features. The computer program code also includes instructions to generate a clinical response prediction to a treatment from the spatial adjacency graph and perform health care treatment actions in response to the clinical response prediction.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram illustrating a high-level system for predicting single-cell spatial genomics from pathology images, in accordance with an embodiment of the present invention;

FIG. 2 is a schematic diagram illustrating an algorithm that can be applied with the single-cell spatial genomics from pathology images, in accordance with an embodiment of the present invention;

FIG. 3 is a schematic diagram illustrating an algorithm that can be applied with the single-cell spatial genomics from pathology images, in accordance with an embodiment of the present invention;

FIG. 4 is a schematic diagram illustrating an algorithm that can be applied with the single-cell spatial genomics from pathology images, in accordance with an embodiment of the present invention;

FIG. 5 is a schematic diagram illustrating an algorithm that can be applied with the single-cell spatial genomics from pathology images, in accordance with an embodiment of the present invention;

FIG. 6 is a schematic diagram illustrating an algorithm that can be applied with the single-cell spatial genomics from pathology images, in accordance with an embodiment of the present invention;

FIG. 7 is a flow diagram illustrating a method for predicting single-cell spatial genomics from pathology images, in accordance with an embodiment of the present invention; and

FIG. 8 is a block diagram illustrating a system for executing single-cell spatial genomics prediction from pathology images, in accordance with an embodiment of the present invention.

### DETAILED DESCRIPTION OF PREFERRED EMBODIMENTS

Technologies such as, e.g., in situ hybridization (ISH) and single-cell sequencing with spatial barcoding allow single-cell level spatial transcriptomics and other genomic data (e.g. proteomics) to be captured along with pathology hematoxylin and eosin stain (H&E) images. Such data provides an informative data source for how cancer patients will respond to treatments, such as, e.g., immunotherapy targeting particular pathways, by allowing important information to be captured about the tumor microenvironment (TME) relevant for discovering biomarkers. However, single-cell spatial genomics analysis methods can be resource-intensive, can be financially expensive, can consume considerable amounts of time to process, can be invasive to collect samples, etc., and cannot be (or is impractical to be) performed routinely for patient screenings.

H&E images can be routinely captured and analyzed by pathologists, resolving several of the issues related to single-cell spatial genomics analysis methods. Being able to impute gene expression from H&E images can be helpful for tasks such as identifying and evaluating biomarkers by avoiding many of the drawbacks of existing single-cell spatial genomics analysis methods. More particularly, imputing spatial gene expression at single-cell resolution is useful for increasing the accuracy of such biomarkers, and can reveal information in greater detail about the development of a tumor, allowing for personalized medical treatment.

Alternatively and additionally to H&E images, other technologies are contemplated to be incorporated in embodiments of the present invention which can include immunohistochemistry (IHC), immunofluorescence (IF), ISH, fluorescence ISH (FISH), periodic acid-Schiff (PAS) stain, Masson's trichrome stain, Alcian blue stain, silver staining, Brightfield microscopy (including unstained tissue), phase-contrast microscopy, differential interference contrast (DIC) microscopy, darkfield microscopy, confocal reflectance microscopy, Quantitative phase imaging (QPI), holographic imaging, multiphoton microscopy, second harmonic generation (SHG) imaging, third harmonic generation (THG) imaging, Raman spectroscopy imaging, infrared (IR) spectroscopy imaging, mass spectrometry imaging (MSI), autofluorescence imaging, virtual staining (e.g., artificial intelligence-based), etc.

Being able to impose sparsity constraints on data from the H&E image can be useful to the training of biomarkers because sparsity allows for the consideration of a smaller number of genes, both for the purpose of helping pathologists to understand the trained model (interpretability); and for comparing response predictions using genomics imputed from image data and predictions generated directly from the ground-truth genomics (which are less expensive to test clinically due to the smaller number of markers used).

Embodiments of the present invention include a joint single-cell spatial genomics and response prediction adaptive spatial graph transformer network (GTN) framework to analyze H&E images. The framework can receive H&E image data associated with N_{data} patients, with a field of view (FOV) selected from a whole slide image, of approximately 0.5×0.5 mm dimensions. Other FOV sizes are also contemplated such as centimeters (cm), micrometers (μm), nanometers (nm), etc. Consequently, individual cells of different cell types can be identified and categorized using spatial transcriptomics at a cellular level.

In some embodiments of the present invention data processed from the H&E images have cells already detected in the FOV, and each cell is associated with image features, such as e.g., morphological features of the cell, nucleus boundary, cell size, cell shape, cell density, cell arrangement, cytoplasmic volume, cytoplasmic staining intensity, cell borders, nuclear size, nuclear shape, nuclear boundary integrity, nuclear-to-cytoplasmic (N:C) ratio, chromatin pattern, hyperchromasia, nuclear pleomorphism, nucleoli prominence, mitotic figures, atypical mitoses, gland formation, keratinization, tumor growth pattern, stromal composition, necrosis, fibrosis/desmoplasia, immune cell infiltration, lymphoid aggregates, vascular density, vascular morphology, edema, hemorrhage, loss of polarity, tissue invasion, irregular nuclear contours, increased mitotic activity, tumor budding, etc.

Additionally, the H&E images can be used to impute, predict, or infer single-cell gene expression, such as, e.g., RNA or protein expression, DNA methylation, histone modifications, chromatin accessibility, chromatin conformation (3D genome structure), transcription factor binding activity, enhancer activity, promoter usage, gene regulatory network activity scores, alternative splicing patterns, RNA isoform usage, RNA velocity, microRNA (miRNA) profiles, long non-coding RNA (lncRNA) profiles, circular RNA (circRNA) profiles, small nucleolar RNA (snoRNA) profiles, copy number variation (CNV), somatic mutation profiles, allele-specific expression patterns, loss of heterozygosity, RNA editing, RNA methylation (e.g., m6A modification), RNA stability differences, metabolic pathway activity scores, spatial gene activity patterns, cell-cell signaling interaction profiles, etc.

The data for each image i∈{1 . . . N_{data}} can be represented by vectors of image features

$${x}_{i}^{c}$$

extracted from each image, where

$${x}_{i}^{c}$$

is the vector associated with the c′th cell associated with patient i. Among the image features, the framework assumes there is an indicator that estimates the cell-type based images features such as e.g., on cell morphology. For instance, categorizing each cell as one of N_{data} categories, such as, e.g., cell type (lymphocyte, macrophage, connective, or tumor).

In some embodiments of the present invention, a spatial adjacency graph Eᵢ⊂{1 . . . Nᵢ}² can be constructed for image i using two thresholds, Tₛₐₘₑ and T_{diff}, where (n, m)∈Eᵢ if d(n,m)≤τₛₐₘₑ and cells n and m have the same type, or d(n, m)≤τ_{diff} and cells n and m have different types, where d(n, m) denotes the Euclidean distance between the centroids of cells n and m. This input data can be formatted as a spatial graph for image i, defined as Gᵢ={Xᵢ, Eᵢ}, where,

$${X}_{i} = [ {x}_{i}^{1} ; … , {x}_{i}^{C} ] ,$$

Xᵢ is a matrix of size Nᵢ×D_{image}, whose c′th row includes the image features from the c′th cell of image i, e.g.,

$${x}_{i}^{c} ,$$

and D_{image} denotes the number of image features (the dimensionality of

$${x}_{i}^{c} ) ,$$

and where [·;·] denotes vertical concatenation of vectors, and.

The framework can associate spatial gene expression data corresponding to each patient, Yᵢ, where Yᵢ can be a matrix of size Nᵢ×D_{gene}, whose c′th row represents the single-cell expression of each gene in cell c (e.g., either an RNA read count, or a protein expression intensity level), and D_{gene} is the number of genes. Additionally, the framework can include data associated with each patient, Rᵢ∈{0,1}, assumed to represent a binary response to treatment (e.g. resistance or responsiveness). The framework can be trained to learn to predict Yᵢ and Rᵢ from Gᵢ.

Accordingly, the framework can have the objective of identifying

$${min}_{θ , S} \sum {ℒ}_{1} ( {\tilde{Y}}_{l} = {ϕ}_{1} ( {X}_{i} ; {E}_{i} ; {θ}_{1} ) , {Y}_{i} ) + {ℒ}_{2} ( {\tilde{R}}_{l} = {ϕ}_{2} ( \text{?} ; {E}_{i} ; {θ}_{2} ) , {R}_{i} ) \qquad ( 1 )$$

$$such that \vert S \vert \leq {τ}_{sparse}$$

where φ(X,E;θ) denotes the output of a graph neural network (GNN) with parameters θ with input node features X and edge matrix E (spatial adjacency graph), S is a set whose size is bounded by the hyperparameter τₛₚₐᵣₛₑ, [symbol] denotes the restriction of \tilde{Y}ₗ to genes in set S, and [symbol] and [symbol] are loss functions, such as a weighted combination of the mean squared error and negative gene-wise spatial Pearson Correlation Coefficient, and the cross-entropy loss respectively. In embodiments of the present invention, φ can have a static GTN architecture. In other embodiments of the present invention, the optimization can minimize the loss of a single loss function such as, e.g., cross-entropy loss.

Other loss functions are also contemplated such as, e.g., mean absolute error, root mean squared error, Huber loss, log-cosh loss, quantile loss, binary cross-entropy loss, categorical cross-entropy loss, sparse categorical cross-entropy loss, Kullback-Leibler divergence, Jensen-Shannon divergence, hinge loss, squared hinge loss, multi-class hinge loss, exponential loss, focal loss, dice loss, Jaccard Loss, Tversky Loss, contrastive loss, triplet loss, cosine embedding loss, center loss, Wasserstein loss, negative log-likelihood, Poisson loss, Cauchy loss, perceptual loss, adversarial loss (generative adversarial network (GAN) loss) etc.

In some other embodiments of the present invention, an adaptive GTN architecture can be implemented using a variant of an adaptive spatial GNN model. The image spatial graph, morphological features, and training data can be selected like those for a static framework, but for the adaptive framework, the GTN can be further associated with an extra layer of weights, W₀, which parameterize a linear projection function of the input features. This can generate a vector of latent meta-features in each cell,

$${z}_{i}^{c} = {x}_{i}^{c} {W}_{0} .$$

For a given W₀, clustering is performed on the meta-features for each cell-type separately using the Leiden algorithm, and the graph Eᵢ is pruned by removing edges between nodes belonging to different clusters of the corresponding cell-types, consequently the refined graph is defined as

$${E}_{i}^{′} = \{ ( {n}_{1} , {n}_{2} ) \vert ( {n}_{1} , {n}_{2} ) ∈ {E}_{i} ∧ \qquad ( 2 )$$

$$( ( {c}_{{n}_{1}} \neq {c}_{{n}_{2}} ) ∨ ( {c}_{{n}_{1}} = {c}_{{n}_{2}} = c ∧ {C}_{c} ( {n}_{1} ) = {C}_{c} ( {n}_{2} ) ) ) \}$$

where cₙ∈{1 . . . N_{type}} is the cell type of cell n, and C_{c}(n) is the cluster assigned to cell n within the clustering for cell-type c. The adaptive cell-level GTN output can then be generated by applying a GTN to the refined graph

$${ϕ}_{1} ( {X}_{1} , {E}_{1} ; {θ}_{GTN} , {W}_{0} ) = GTN ( {X}_{1} , {E}_{i}^{′} ; {θ}_{GTN} ) \qquad ( 3 )$$

which assumes, for the adaptive model, θ=(θ_{GTN}, W₀). The model can then be trained by applying variational optimization to both the adaptive GTN parameters and the sparsity constraints. Additionally, the genomics imputation model can be pretrained, and can then be frozen during the optimization of the response model, or the two models can be jointly optimized using Equation (1).

The parameters W₀, the sparse gene-set S, and the remaining parameters of the GTN can be trained jointly, using variational optimization. This can be achieved by introducing an auxiliary variable, ψ∈ [symbol] , and letting:

S_{ψ}=(g;ψ_{g}>0) (4)

The optimization of Equation (1) can be equivalent to a minimization problem over θ′={θ,ψ}. The variational distribution over θ′ can include

Q_{μ,σ}(θ′)= [symbol] (θ,μ,σ) (5)

where μ is a real vector of length len(W₀(:))+len(θ_{GTN}(:))+N_{gene}, where (:) represents vectorization, and len is vector length, and σ is a scalar. Additionally, the variational objective function can be employed

$$F ( {θ}^{′} = \{ θ , ψ \} ) = \{ \begin{matrix}( \frac{1}{D} ) {\sum }_{i} - ℒ ( {X}_{i} , {E}_{i} , {Y}_{i} , {R}_{i} ) + O , if \vert {S}_{ψ} \vert \leq {ψ}_{sparse} \\ 0 , otherwise\end{matrix} \qquad ( 6 )$$

where [symbol] (Xᵢ, Eᵢ, Yᵢ, Rᵢ) denotes the combined loss defined in Equation (1) for GTNs trained on input graph (Xᵢ, Eᵢ), D is the number of datapoints, and O is a constant offset,

$$O = {min}_{θ , ψ , i} ℒ ( {X}_{i} , {E}_{i} , {Y}_{i} , {R}_{i} ) ,$$

which ensures F(θ) is non-negative. Consequently, assuming F(θ) is not the constant 0 function, for any solution

$${θ}^{′} = \{ θ , ψ \} ∈ {arg max}_{{θ}^{′}} F ( {θ}^{′} ) ,$$

(θ, S_{ψ}) is a solution to Equation (1).

Further, the variational bound

$${max}_{μ , σ} {𝔼}_{θ ′ ∼ {Q}_{μ , σ}} [ F ( {θ}^{′} ) ] \leq {max}_{{θ}^{′}} F ( {θ}^{′} ) \qquad ( 7 )$$

and can optimize Equation (1) by performing variational optimization over θ in some embodiments of the present invention, for instance by using the smoothing-based optimization updates

$${μ}_{t + 1} = \frac{{Σ}_{S} {f}_{S}^{t} {θ}_{S}^{′t}}{{Σ}_{S} {f}_{S}^{t}} , \qquad ( 8 )$$

$${σ}_{t + 1} = \sqrt{\frac{{Σ}_{S} {f}_{S}^{t} {\vert {θ}_{S}^{′ t} - {μ}_{t} \vert}_{2}^{2}}{P {Σ}_{S} {f}_{S}^{t}}}$$

for s∈{1 . . . S} are samples drawn from Qₜ= [symbol] (·; μₜ, σₜ), and

$${f}_{S}^{t} = F ( {θ}_{S}^{′ t} ) .$$

The updates in Equation (8) can increase

$${𝔼}_{{θ}^{′} ∼ {Q}_{μ , σ}} [ F ( {θ}^{′} ) ]$$

in expectation.

The framework defined in Equation (1) can be trained jointly, e.g., the parameters of the genomics imputation and response prediction GTNs, along with W₀ and sparse gene selection φ parameters can be jointly optimized. Alternatively, the gene expression imputation GTN can first be optimized, and these parameters can then be frozen, and the gene selection φ parameters and response prediction GTN can then be optimized subsequently. In each case, the adaptive GTN parameters W₀ can be tied between the GTNs or learned separately. Training in a piecewise configuration can have several benefits, including faster training, more stable model, more varied degrees of supervision, and less complex optimization.

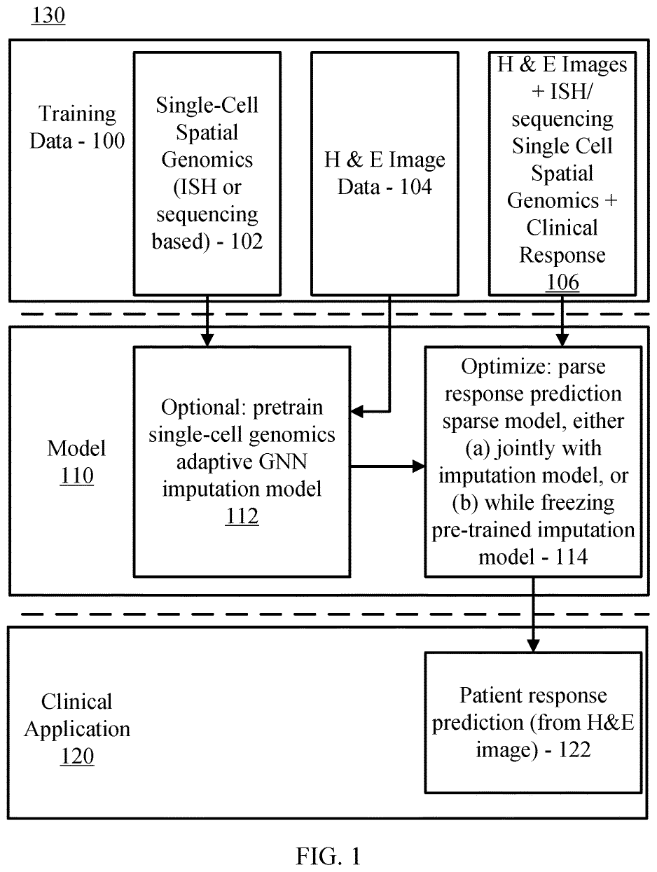

Referring now in detail to the figures in which like numerals represent the same or similar elements and initially to FIG. 1, a flow diagram illustrating an exemplary use of embodiments of the present invention is illustrated. Framework 130 can predict single-cell spatial genomics from pathology images for patient stratification. Framework 130 can be an artificial intelligence (AI) model, having several components including training data 100, model 110, and clinical operation 120. Training data 100 can include single-cell spatial genomics 102. The genomics can be ISH or sequencing based. Additionally, there can be H&E image data 104. Further, there can be a combination of H&E images 104 and ISH/sequencing single cell spatial genomics and a reported/documented clinical response 106.

Model 110 can receive single-cell spatial genomics 102 and H&E image data 104 as an optional embodiment of the present invention. Single-cell spatial genomics 102 and H&E image data 104 are received by pretrain single-cell genomics adaptive GNN imputation model 112. Model 110 can also be optimized to parse response prediction sparse model 114 which can be performed jointly with imputation model 112, or while freezing pre-trained imputation model 112.

Once response prediction sparse model 114 optimizes documented clinical response 106 with, or without adaptive GNN imputation model 112, the patient response prediction 122 can be generated from H&E image data 104. Patient response prediction 122 can be a clinical application 120 of the framework 130.

Patient response prediction 122 can automatically trigger treatment recommendations and preparation. In some embodiments of the present invention, response prediction sparse model 114 can be incorporated into an agentic agent for developing plans for further testing, chemotherapy, radiation, or other treatment options; generating lists of symptoms and conditions to consider for treatments; scheduling appointments; contacting appropriate personnel (e.g., family, medical professionals, etc.); etc. Additionally patient response prediction 122 can result in a recommendation of ceasing treatment or avoiding future treatment. Since chemotherapy, radiation, and other treatments can be financially, physically, and emotionally taxing as well as wasteful of resources if not used properly, identifying whether one or more treatments will be effective and whether the treatments are necessary for the patient can be of value.

The treatment recommendations and preparation can also include drafting documents, documentation of the testing, records, auditing/validating/storing data, ensuring regulatory compliance of the privacy and security of the data, etc.

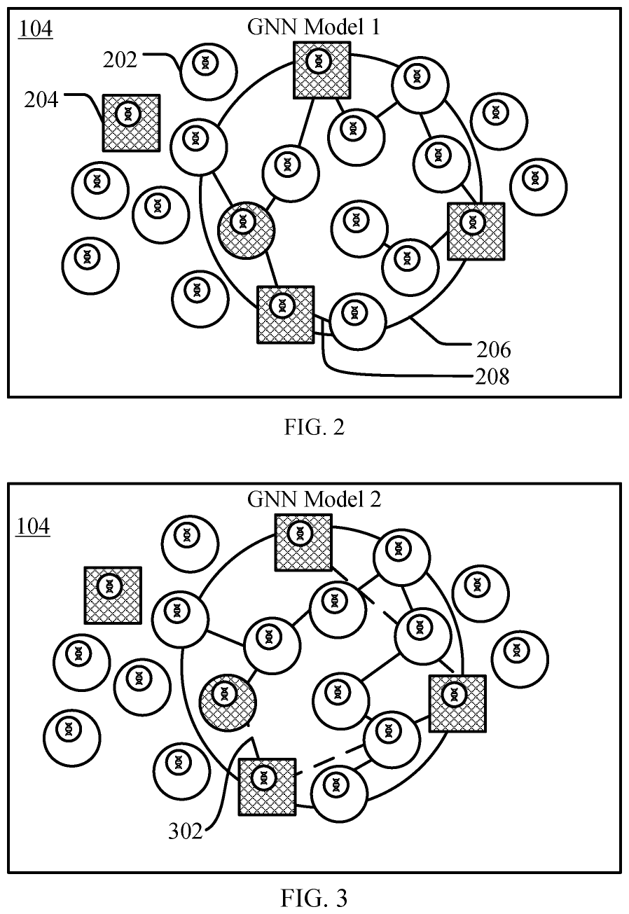

Referring to FIG. 2, an alternative embodiment of the present invention for identifying and separating data in H&E images 104 is illustrated. GNN model 1 in FIG. 2 includes questionable cells 204, normal cells 202, edge 208, and radius 206. FIG. 2 illustrates a simplified H&E image 104 that can be processed by adaptive GNN imputation model 112 and 114 (FIG. 1). Normal cells 202 which include cell features, e.g., cell shape, which are characterized as normal. Questionable cells 204 can raise concerns for further investigation or evaluation to determine their classification as cancerous or not. H&E image 104 can look at a portion, e.g., a limited FOV. The FOV can be radius 206. The FOV can also be rectangular, square, or another shape.

All cells within radius 206 are connected with edge 208. If any portion of normal cells 202 or questionable cells 204 is within radius 206, then the cell is connected with edge 208. Edge 208 can be used to form a graph for interpretation of spatial transcriptomics, by identifying genes as features of each cell in the graph formed by the connections represented by edge 208.

Referring to FIG. 3, an alternative embodiment of the present invention for identifying and separating data in H&E images 104 is illustrated. GNN model 2, as depicted in FIG. 3, is an alternative to GNN model 1, where cells of a same type are connected. In other words, while GNN model 1 connects all cells within a particular spatial distance, while GNN model 2 connects all cells of the same type within a particular spatial distance. Abnormal edge 302 can connect all questionable cells 204 while edge 208 is for normal cells 202. The spatial distance, density, frequency, degree of gene expression, etc. can be measured and considered by the framework.

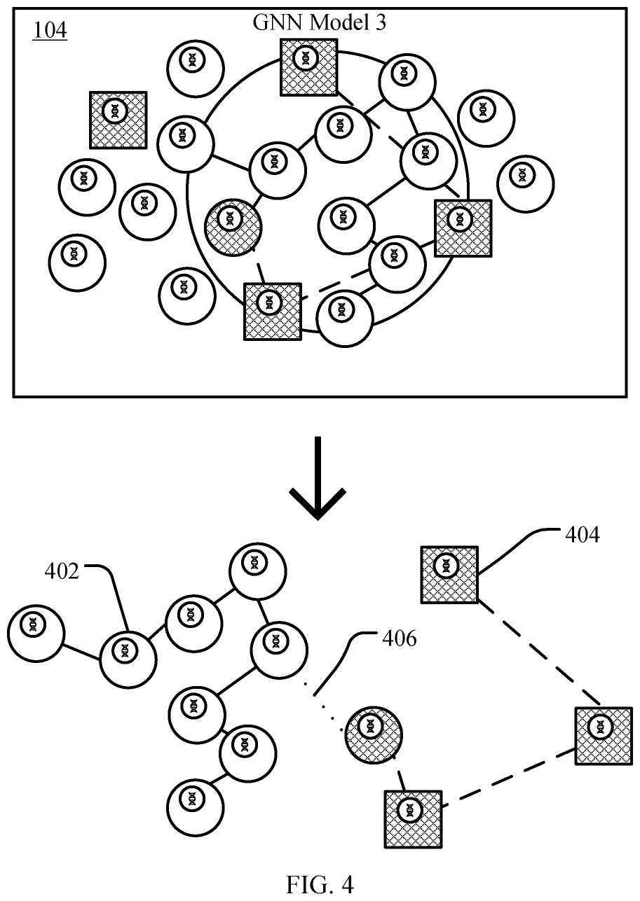

Referring to FIG. 4, an alternative embodiment of the present invention for identifying and separating data in H&E images 104 is illustrated. GNN model 3 in FIG. 4 is a modified version of GNN model 2. GNN model 3 connects all cells of a same type within a particular spatial distance, then clusters cells of each type and prunes edges between clusters. The edge to be pruned can be cluster edge 406 which separates normal cluster 402 from questionable cluster 404. Normal cluster 402 and questionable cluster 404 can be connected by questionable cells 204 that have some questionable features (as seen by the hatch pattern), but some features of normal cells 202 (as seen by the circular shape). Cluster edge 406 can be pruned to separate normal cluster 402 and questionable cluster 404 and is depicted to illustrate a potential connection between the clusters. Cluster edge 406 is depicted to illustrate connections, however cluster edge 406 is pruned in some embodiments of the present invention.

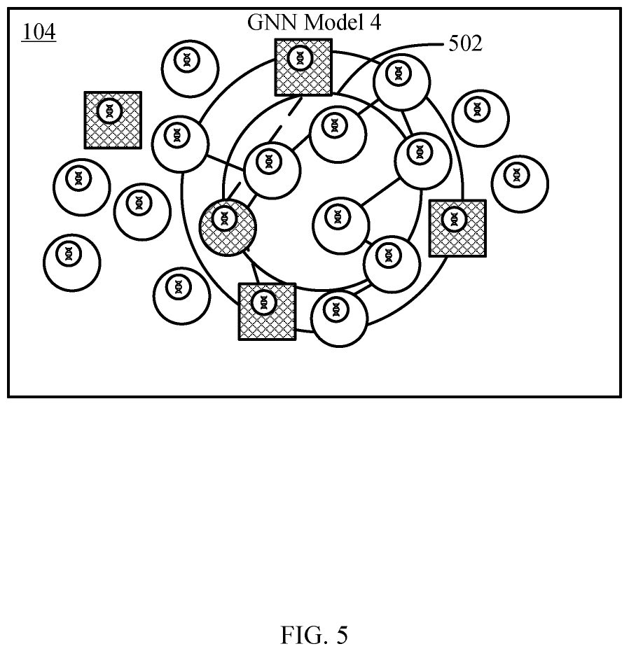

Referring to FIG. 5, an alternative embodiment of the present invention for identifying and separating data in H&E images 104 is illustrated. GNN model 4 of FIG. 5 connects all cells of the same types within a distance d₁ (radius 206), and all cells of different types within d₂ (inner radius 502), where d₁>d₂. GNN model 4 is similar to GNN model 2, however there is a spatial distance threshold for cells of a different type. Questionable cells 204 have a smaller cluster since less cells are within inner radius 502 than are in radius 206. This can have the model focus on spatial proximity more so than the other GNN models.

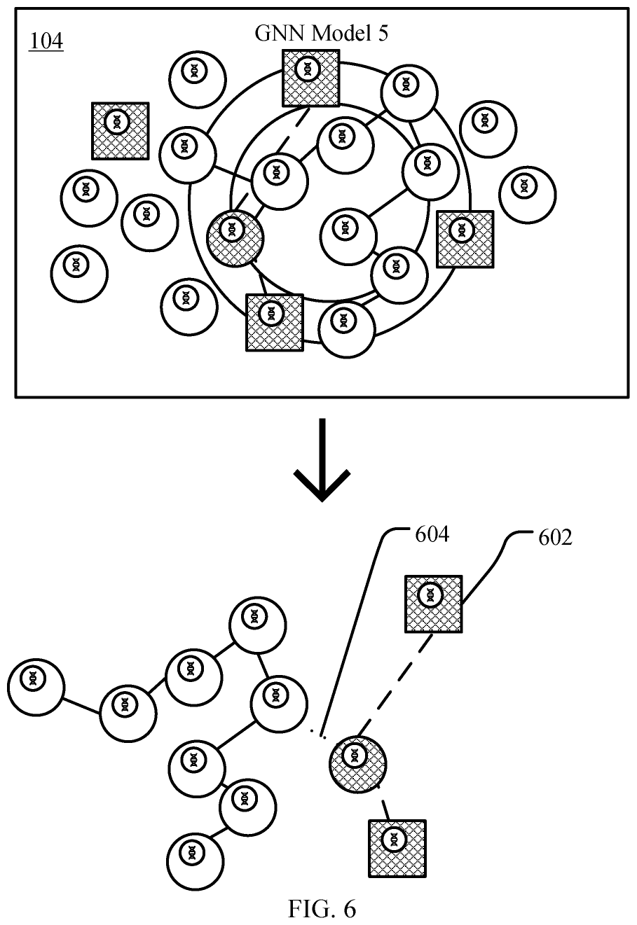

Referring to FIG. 6, an alternative embodiment of the present invention for identifying and separating data in H&E images 104 is illustrated. GNN model 5 of FIG. 6 applies GNN model 4 and further clusters cells of each type and prunes same-same edges between clusters like GNN model 3. Minimized questionable cluster 602 is similar to questionable cluster 404. Minimized cluster edge 604 is similar to cluster edge 406. Minimized cluster edge 604 is depicted to illustrate connections, however minimized cluster edge 604 is pruned in some embodiments of the present invention.

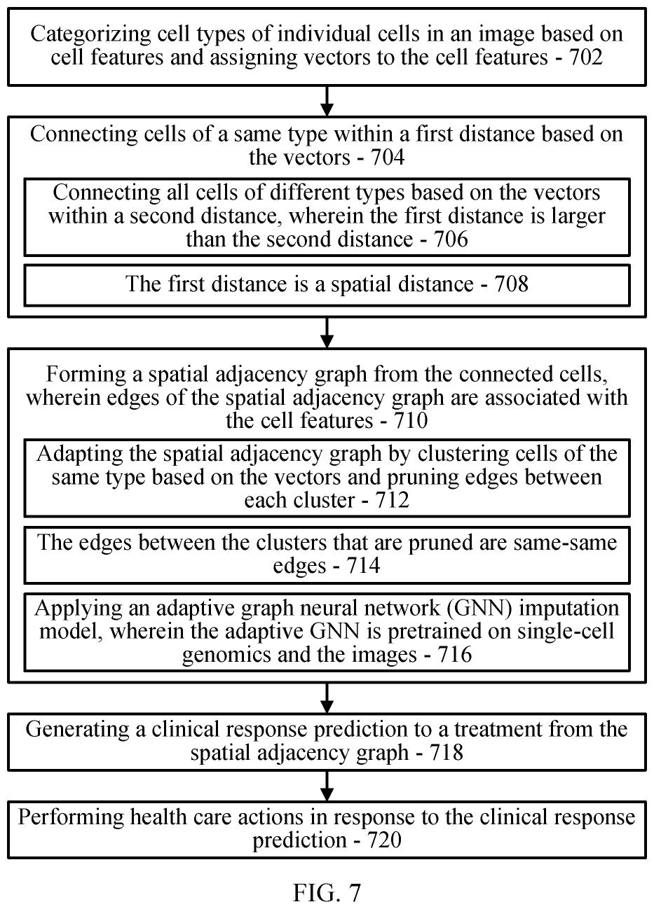

Referring to FIG. 7, a flow diagram illustrating a method of predicting single-cell spatial genomics from pathology images for patient stratification is illustratively depicted. Some of the blocks can be optional and multiple embodiments of the present invention can be derived from various combinations of the blocks. In block 702, cell types of individual cells in an image are categorizing based on cell features and assigning vectors to the cell features. The type of cell can be based on cell features, such as shape, size, circularity, cell identification, cytoplasm volume, nuclear size, tissue architecture, etc. In block 704, cells of a same type with a first distance are connected based on the vectors.

In optional block 706, all cells of different types are connected based on the vectors within a second distance, wherein the first distance is larger than the second distance. In optional block 708, the distance can be a spatial distance such as Euclidean distance, Manhattan distance, or Chebyshev distance. The distance can be from each cell centroid. The distance can also be cosine similarity, graph distance, statistical distance (e.g., Kullback-Leibler divergence, Jensen-Shannon divergence, Wasserstein distance, etc.), etc.

In block 710, a spatial adjacency graph is formed from the connected cells, wherein edges of the spatial adjacency graph are associated with the cell features. In optional block 712, the spatial adjacency graph is adapted by clustering cells of the same type based on the vectors and pruning edges between each cluster. In optional block 714, edges between clusters are pruned. The edges can be same-same edges. Cells that have the same features can be assigned to different clusters if other features vary, but still be connected with same-same edges, unless pruned. Determining which cluster a cell belongs to can occur in several ways. The graph can interrelate any number of cell features. The edge weights can be assigned and changed by a user. The edges can be directed or undirected. Edges can be normalized.

In optional block 716, an adaptive graph neural network (GNN) imputation model is applied to form the spatial adjacency graph, wherein the adaptive GNN is pretrained on single-cell genomics and H&E images. In block 718, a clinical response prediction is generated to a treatment from the spatial adjacency graph. In block 720, health care actions are performed in response to the clinical response prediction. The health care action can be making a record of the prediction or identification of certain features in the image such as, e.g., cancerous cells; notifying doctors, nurses, family members, etc., of the prediction or other information; develop a treatment plan; begin ordering supplies, surgeries, drafting documentation for treatment and insurance approval, scheduling follow up appointments, etc. Additionally, health care actions can include determining that future action is not useful/productive, indicating future health care actions can cease. Other types of healthcare treatment are also contemplated.

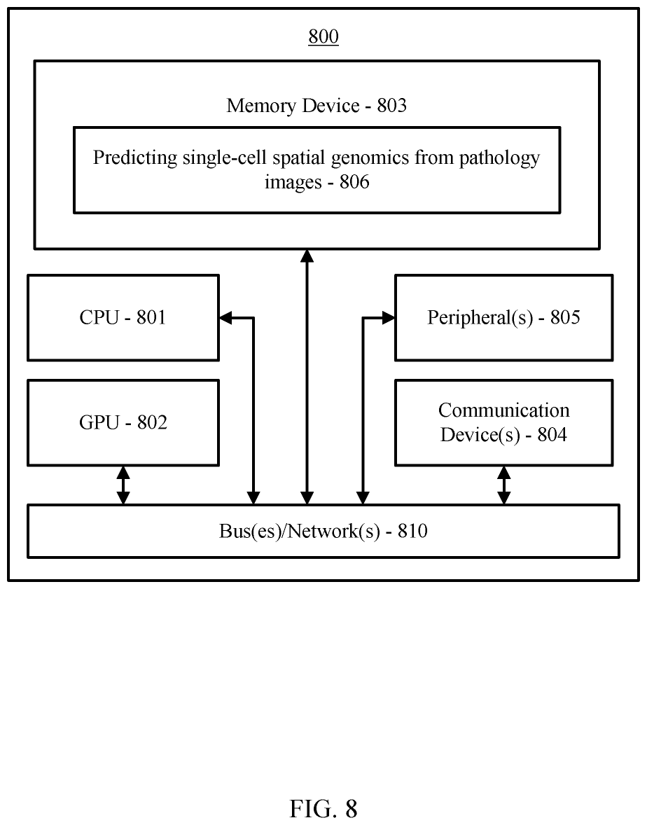

Referring to FIG. 8, a block diagram is shown for an exemplary processing system 800, in accordance with an embodiment of the present invention. The processing system 800 includes a set of processing units (e.g., CPUs) 801, a set of GPUs 802, a set of memory devices 803, a set of communication devices 804, and a set of peripherals 805. The CPUs 801 can be single or multi-core CPUs. The GPUs 802 can be single or multi-core GPUs. The one or more memory devices 803 can include caches, RAMs, ROMs, and other memories (flash, optical, magnetic, etc.). The communication devices 804 can include wireless and/or wired communication devices (e.g., network (e.g., Wi-Fi®, etc.) adapters, etc.). The peripherals 805 can include a display device, a user input device, a printer, an imaging device, and so forth. Elements of processing system 800 are connected by one or more buses or networks (collectively denoted by the figure reference numeral 810).

In an embodiment of the present invention, memory devices 803 can store specially programmed software modules to transform the computer processing system into a special purpose computer configured to implement various embodiments of the present invention. In an embodiment, special purpose hardware (e.g., Application Specific Integrated Circuits, Field Programmable Gate Arrays (FPGAs), and so forth) can be used to implement various embodiments of the present invention.

In an embodiment, memory devices 803 store program code or software 806 for predicting single-cell spatial genomics from pathology images. The software 806 implements one or more functions of the systems and methods described herein for categorizing cell types of individual cells in an image based on cell features and assigning vectors to the cell features, connecting cells of a same type within a first distance based on the vectors, and forming a spatial adjacency graph from the connected cells, wherein edges of the spatial adjacency graph are associated with the cell features. Software 806 also includes generating a clinical response prediction to a treatment from the spatial adjacency graph and performing health care actions in response to the clinical response prediction.

Of course, the processing system 800 may also include other elements (not shown), as readily contemplated by one of skill in the art, as well as omitting certain elements. For example, various other input devices and/or output devices can be included in processing system 800, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 800 are readily contemplated by one of ordinary skill in the art given the teachings of the present invention provided herein.

Moreover, it is to be appreciated that various figures as described with respect to various elements and steps relating to the present invention that may be implemented, in whole or in part, by one or more of the elements of system 800.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs). These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment,” as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method comprising:
categorizing cell types of individual cells in an image based on cell features and assigning vectors to the cell features;
connecting cells of a same type within a first distance based on the vectors;
forming a spatial adjacency graph from the connected cells, wherein edges of the spatial adjacency graph are associated with the cell features;
generating a clinical response prediction to a treatment from the spatial adjacency graph; and
performing health care actions in response to the clinical response prediction.

2. The method of claim 1, further comprising:
adapting the spatial adjacency graph by:
clustering cells of the same type based on the vectors; and
pruning edges between each cluster.

3. The method of claim 1, further comprising:
connecting all cells of different types based on the vectors within a second distance, wherein the first distance is larger than the second distance.

4. The method of claim 3, further comprising:
adapting the spatial adjacency graph by:
clustering cells of each type; and
pruning edges between each cluster.

5. The method of claim 4, wherein the edges between the clusters that are pruned are same-same edges.

6. The method of claim 1, wherein generating the clinical response prediction further includes;
applying an adaptive graph neural network (GNN) imputation model, wherein the adaptive GNN is pretrained on single-cell genomics and the images.

7. The method of claim 1, wherein the first distance is a spatial distance.

8. A system, comprising:
a processor; and
a memory storing computer-readable instructions that, when executed by the processor, cause the processor to:
categorize cell types of individual cells in an image based on cell features and assigning vectors to the cell features;
connect cells of a same type within a first distance based on the vectors;
form a spatial adjacency graph from the connected cells, wherein edges of the spatial adjacency graph are associated with the cell features;
generate a clinical response prediction to a treatment from the spatial adjacency graph; and
perform health care actions in response to the clinical response prediction.

9. The system of claim 8, wherein the memory further causes the processor to:
adapt the spatial adjacency graph by:
cluster cells of the same type based on the vectors; and
prune edges between each cluster.

10. The system of claim 8, wherein the memory further causes the processor to;
connect all cells of different types based on the vectors within a second distance, wherein the first distance is larger than the second distance.

11. The system of claim 10, wherein the memory further causes the processor to;
adapt the spatial adjacency graph by:
cluster cells of each type; and
prune edges between each cluster.

12. The system of claim 11, wherein the edges between the clusters that are pruned are same-same edges.

13. The system of claim 8, wherein causing the processor to generating the clinical response prediction further includes causing the processor to;
apply an adaptive graph neural network (GNN) imputation model, wherein the adaptive GNN is pretrained on single-cell genomics and the images.

14. The system of claim 8, wherein the first distance is a spatial distance.

15. A computer program product comprising a non-transitory computer-readable storage medium containing computer program code, the computer program code when executed by one or more processors causes the one or more processors to perform operations, the computer program code comprising instructions to:
categorize cell types of individual cells in an image based on cell features and assigning vectors to the cell features;
connect cells of a same type within a first distance based on the vectors;
form a spatial adjacency graph from the connected cells, wherein edges of the spatial adjacency graph are associated with the cell features;
generate a clinical response prediction to a treatment from the spatial adjacency graph; and
perform health care actions in response to the clinical response prediction.

16. The computer program product of claim 15, wherein the one or more processors:
adapt the spatial adjacency graph by:
cluster cells of the same type based on the vectors; and
prune edges between each cluster.

17. The computer program product of claim 15, wherein the one or more processors;
connect all cells of different types based on the vectors within a second distance, wherein the first distance is larger than the second distance.

18. The computer program product of claim 17, wherein the one or more processors;
adapt the spatial adjacency graph by:
cluster cells of each type; and
prune edges between each cluster.

19. The computer program product of claim 18, wherein the edges between the clusters that are pruned are same-same edges.

20. The computer program product of claim 15, wherein when the one or more processors generate the clinical response prediction, the one or more processors;
apply an adaptive graph neural network (GNN) imputation model, wherein the adaptive GNN is pretrained on single-cell genomics and the images.
